(19) United States
(12) Patent Application Publication
(10) Pub. No.: US 2011/0177137 A1
(43) Pub. Date: Jul. 21, 2011

(54) NOVEL DOSAGE FORM OF PALIPERIDONE AND PROCESS FOR PREPARING THE SAME

(75) Inventors: Vijaysinh Vanvirsinh Chauhan, Ahmedabad (IN); Satyavan Shivajirao Dhavale, Ahmedabad (IN); Sanjay Maganbhai Patel, Ahmedabad (JP); Jayanta Kumar Mandal, Ahmedabad (IN); Kirti Bansidhar Maheshwari, Ahmedabad (IN)

(73) Assignee: INTAS PHARMACEUTICALS LIMITED, Ahmedabad. Gujarat (IN)

(21) Appl. No.: 13/063,836

(22) PCT Filed: Sep. 14, 2009
(86) PCT No.: PCT/IN09/00503

§ 371 Date: Apr. 4, 2011

(30) Foreign Application Priority Data
Sep. 15, 2008 (IN) 1958/MUM/2008

(51) Int. Cl. A61K 31/519; A61K 9/00; A61P 25/18; C09D 171/08; B05D 3/12

(52) U.S. Cl. 424/400; 514/259.41; 524/612; 427/2.21

(57) ABSTRACT

The present invention relates to an extended release composition of paliperidone for oral administration comprising paliperidone and at least one matrixing agent. The said extended release composition maintains desired therapeutic drug effect over a prolonged period of time and thereby reduces the side effects resulting due to excess drug blood plasma concentration. Further, the invention also relates to process for the preparation of an extended release oral composition of paliperidone.

### FIELD OF THE INVENTION

This invention relates to an extended release composition of paliperidone for maintaining desired therapeutic drug effect over a prolonged period of time within gastrointestinal track in an extended release manner and thereby reducing the side effects resulting due to excess drug blood plasma concentration.

### BACKGROUND AND PRIOR ART

The present invention describe with an extended release of paliperidone, a hydroxyl derivative of risperidone. Paliperidone is insoluble in water, partly soluble in methyl chloride and soluble in 0.1 N HCl. It has a long half-life of about one day and degrades into detectable amount of impurities like C-9 ketoes, N-Oxides, and dimmers. Further high blood plasma concentration of paliperidone restricts its immediate release dosage administration as high concentration of drug in blood plasma produces several side effects like anxiety, somnolence, dizziness, constipation, and extrapyrimidal symptoms.

U.S. Pat. No 5,158,952 claims paliperidone or its pharmaceutically acceptable acid addition salts. Further it also claims for method of treating warm-blooded animals suffering from psycotic disease.

U.S. Pat. No. 5,536,507 discloses enteric coated pellets comprising a core of active ingredient, microcrystalline cellulose, pH sensitive polymer and optionally osmotic agent wherein core is coated with non-water soluble polymers followed by enteric coat. The disclosed compositions mainly applicable to active substances, which are unstable in the lower pH range of gastrointestinal tract, can cause stomach irritation or weak bases or salts.

WO 2004010981 & WO 2006085856 discloses osmotic system to achieve extended release of paliperidone. This system utilize osmotic pressure to generate driving force for imbibing fluid into a compartment formed by a semi permeable membrane that permits free diffusion of fluid but not drug or osmotic agent. This pH independent system comprising semi permeable membrane surrounding three-layer core, wherein first drug layer, adjacent to an orifice drilled through the membrane, comprising low amount of drug and osmotic agents; middle layer containing higher amount of drug, excipients and no salt and third push layer, contains osmotic agents and no drug. Further these patents also disclose the method of release of drug through the said composition.

WO 2006017537 discloses double matrix layered extended release dosage form wherein first delay layer comprising matrix polymer and microencapsulated drugs, free from non-microencapsulated drug while other layer comprising polymer matrix and non-microencapsulated drug and both the layers are adjacent to each other. This patent has focused to micro encapsulation of drug that is benefited to slow release of drug compared to non-microencapsulated drug.

The inventors of the present invention have tried to achieve extended release of paliperidone through conventional economic process by incorporating matrixing agent intragranularly and extra granularly and functional coat of pH independent polymer.

### OBJECT OF THE INVENTION

The main object of the invention is to attain extended release of paliperidone by making dosage form in conventional way that is benefited with respect to economy and reduction of time consumption to industry.

Another object of the invention is to develop extended release composition that control the blood plasma concentration of a drug and thereby prevent the side effects occurred due to high blood plasma concentration of drug.

Still one more object of the invention is to provide extended release oral composition having more retentive and complete release of an active ingredient after its administration.

Still another object of the invention is to provide a process for preparing extended release composition by incorporating matrixing agent intragranularly and extra granularly in conventional way.

Still one more object of the invention is to attain at least 50% of the release of active ingredient in 12 hours.

### SUMMARY OF THE INVENTION

The present invention is directed towards extended release solid oral composition comprising a core, inclusive of intragranular-extragranular application of matrixing agent, comprising active ingredient, one or more polymer matrix and one or more pharmaceutically acceptable excipients wherein granules are subject to compression followed by functional coat of pH independent polymer.

Further the present invention is also directed toward the process for preparation of extended release composition of paliperidone as describe below.

### DETAILED DESCRIPTION OF THE INVENTION

In one of the embodiment of the present invention is compressed solid dosage form, provided for extended release of drug.

The composition of the present invention includes two compartments: First compartment, the core, is manufactured by use of matrixing agent intragranularly and extra granularly wherein the other intragranular ingredients comprises paliperidone or its pharmaceutically acceptable salts, one or more matrixing agent and other pharmaceutically acceptable excipients and extragranuler comprises one or more matrixing agent and/or other pharmaceutically acceptable excipients.

The second compartment of the composition, functional coat, comprises low permeable polymer and other acceptable excipients. Composition may also be further coated with color coat for aesthetic appeal.

The active ingredient in the present composition is ranged from 1-20% w/w of the composition.

Matrixing agent used in both intragranular as well as extragranuler, in the range of 1-80%, includes natural or synthetic are selected from the group comprising polysaccharides such as xanthan, pullulan, chitosan and the like; Gums like guar gum, gum arabic, gum karaya, and the like; and cellulose ethers, such as hydroxymethylcellulose (HMC), hydroxyethylcellulose (HEC), hydroxypropylcellulose (HPC), methylcellulose (MC), ethylcellulose (EC), Carboxyethylcellulose (CEC), ethylhydroxyethylcellulose (EHEC), Carboxymethylhydroxyethylcellulose (CMHEC), hydroxypropylethylcellulose (HPEC), Hydroxypropyl methyl cellulose (HPMC) and sodium carboxymethylcellulose (Na CMC); Polymeric methacrylates; Carbomers as well as copolymers and/or mixtures of any of the above polymers, provided that matrixing agent characterized variant water permeability within intragranuler and extragranular.

The pharmaceutically acceptable excipients within the intragranular as well as extragranuler of the core may include diluent, binder, glidant, lubricant, antioxidant, solvents or mixtures thereof.

Diluent in core, ranges from 5 to 95% w/w of the composition, is selected from the group comprising but not limited to lactose, sucrose, mannitol, sorbitol, maltodextrin, erythritol, powdered cellulose, microcrystalline cellulose, silicified microcrystalline cellulose, starch, dibasic calcium phosphate anhydrous, tribasic calcium phosphate, kaolin, precipitated calcium carbonate.

Binders in the core, ranges from 1-25% w/w of the composition, is selected from the group comprising but not limited to povidone, hydroxypropylmethylcellulose, acacia, starch, alginic acid, hydroxyethylcellulose, carboxymethylcellulose sodium, sugar, gelatin, liquid glucose, methyl cellulose, pregelatinized starch and the like

Antioxidant in the core, ranges from 0.05-2% w/w of the composition, is selected from the group comprising butylated hydoxyl anisol(BHA), Butylated hydroxyl Toluene (BHT), Vitamin E and the like

Solvents, used for the preparation of binding solution, are selected from the group comprising but not limited to water, isopropyl alcohol, ethanol, methanol, acetone, acetonitril, methylenechloride, ether, nucleotides, chloroform, 1,4-dioxane, tetrahydrofuran, dimethyl sulfoxide, ethylacetate, methylacetate or mixtures thereof;

Lubricant in the core, ranges from 0.5-10% w/w of the composition, is selected from the group comprising but not limited to stearic acid, polyethylene glycol, magnesium stearate, calcium stearate, talc, zinc stearate, hydrogenated castor oil, silica, colloidal silica, cornstarch, calcium silicate, magnesium silicate, silicon hydrogel and the like.

Further functional coat of pH independent polymer, ranges of 2-20% w/w of the core, comprises low water permeable polymers, plasticizers, opacifiers, colorants and other suitable excipients.

Low water permeable polymer, having an active character in extended release of active ingredient, are selected from the group comprising but not limited to co-polymers of acrylic and methacrylic acid esters like Eudragit RL, ethyl cellulose, prolamine, polyethylene oxide, polyvinyl acetate, zein.

Plasticizers range from 5 to 50% w/w of film forming polymer. Plasticizers can be selected from polyethylene glycol, acetyl triethyl citrate, acetyl tributyl citrate, triethyl citrate, acetylated monoglycerides, glycerol, triacetin, propylene glycol, dibutyl phthalate, diethyl phthalate, isopropyl phthalate, dimethyl phthalate, dactyl phthalate, dibutyl sebacate, dimethyl sebacate, castor oil, glycerol monostearate, fractionated coconut oil, others or a combination thereof.

Opacifiers, ranges from 8-25% of the coat, include water insoluble pigments comprising titanium dioxide, calcium carbonate, calcium sulfate, magnesium oxide, magnesium carbonate, aluminum silicate, aluminum hydroxide, talc and iron oxide.

Colorants, ranges from 0.05-8% w/w of coat, include water soluble dyes, water insoluble pigments and natural colorants.

Another embodiment of the present invention includes process for preparation of extended release pharmaceutical composition of paliperidone by incorporating matrixing agent intragranularly and extra granularly.

The above process mainly includes three steps from which First step, intragranulation by wet granulation, comprising granulation of geometric mixture of API, diluents and matrixing agent with binder solution followed by drying and sieving to get dry granules. Second step, extra granulation, comprising incorporating matrixing agent and lubricant to dry granules obtained through the first step followed by compression. Finally in Third step, functional coating of pH independent polymer to compressed dosage form obtained in second step.

The present invention as classify serves in attaining extended release of paliperidone or its pharmaceutically acceptable salts wherein at least 50% of the active ingredient is release within 12 hours.

The extended release pharmaceutical composition of the present invention is in a solid dosage form as a monolithic system, multi-particulate system, matrix system, matrix with coating system and the likes thereof.

Throughout this specification and the appended claims it is to be understood that the words “comprise” and include” and variations such as “comprises”, “comprising”, “includes”, “including” are to be interpreted inclusively, unless the context requires otherwise. That is, the use of these words may imply the inclusion of an element or elements not specifically recited.

### EXAMPLE

The present invention has been described by way of example only, and it is to be recognized that modifications thereto falling within the scope and spirit of the appended claims, and which would be obvious to a person skilled in the art based upon the disclosure herein, are also considered to be included within the scope of this invention. The above said invention can be illustrated by but not limited to following example(s):

### Example 1

| Sr. No | Ingredients | % w/w |
|---|---|---|
| 1 | Paliperidone | 1.93 |
| 2 | Lactose | 44.8 |
| 3 | HPMC K-4M | 32.1 |
| 4 | Povidone K-30 | 3.85 |
| 5 | Isopropyl alcohol | Qs |
| 6 | Purified water | Qs |
| 7 | HPMC K 100 LV | 12.3 |
| 8 | Stearic acid (60#) | 0.97 |
| 9 | BHA | 0.19 |
| | Functional Coating | |
| 1 | PEG 4000 | 0.52 |
| 2 | Isopropyl alcohol | Qs |
| 3 | Dichloromethane | Qs |
| 4 | Eudragit RSPO | 3.34 |

### Process for Preparation

### A) Intragranular Application of Matrixing Agent

1) Weigh and sift Paliperidone, Lactose and HPMC K-4M trough 40#.

2) Mix step 1) carefully and ensures geometric mixing.

3) Dissolve Povidone K-30 in isopropyl alcohol: purified water mixture.

4) Granulate step 2) with binding solution of step 3).

5) Dry the granules and pass the dried granules through 20#.

### B) Extragranular Application of Matrixing Agent

6) Weigh and sift HPMC K 100 LV, Stearic acid (60#) and BHA through 40#.

7) Lubricate step 5) with step 6). Mix well for 5 minutes.

8) Compress the resultant blend of step 7)

### C) Coating by pH Independent Polymer

1) Dissolve PEG 4000 in isopropyl alcohol: dichloromethane solution with stirring.

2) Add Eudragit RSPO in step 1) with stirring.

3) Mix the solution for 10-20 minutes with stirring.

4) Pass the solution through 200#.

5) Perform the coating with the coating solution of step 4)

### Example 2

| Sr. No | Ingredients | % w/w |
|---|---|---|
| 1 | Paliperidone | 3.73 |
| 2 | Lactose | 41.61 |
| 3 | HPMC K-4M | 31.06 |
| 4 | Povidone K-30 | 3.73 |
| 5 | Isopropyl alcohol | Qs |
| 6 | Purified water | Qs |
| 7 | HPMC K 100 LV | 11.92 |
| 8 | Stearic acid (60#) | 0.93 |
| 9 | BHA | 0.19 |
| | Functional Coating | |
| 1 | PEG 4000 | 0.86 |
| 2 | Isopropyl alcohol | Qs |
| 3 | Dichloromethane | Qs |
| 4 | Eudragit RSPO | 0.78 |
| 5 | Titanium dioxide | 0.86 |
| 6 | Ferric oxide red | 0.12 |

### Process for Preparation

### A) Intragranular Application of Matrixing Agent (as Per Example 1)

### B) Extragranular Application of Matrixing Agent (as Per Example 1)

### C) Coating by pH Independent Polymer

1) Dissolve PEG 4000 in approx 75% quantity of isopropyl alcohol: dichloromethane mixture with stirring.

2) Add Eudragit RSPO in step 1) with stirring.

3) Disperse the titanium dioxide and ferric oxide red in remaining quantity of isopropyl alcohol: dichloromethane mixture.

4) Mix the solution of step 2) and 3) for 10-20 minutes with stirring.

5) Pass the solution through 200#

6) Perform the coating with the coating solution of step 5)

### Example 3

| Sr. No | Ingredients | % w/w |
|---|---|---|
| 1 | Paliperidone | 5.59 |
| 2 | Lactose | 39.76 |
| 3 | HPMC K-4M | 31.01 |
| 4 | Povidone K-30 | 3.73 |
| 5 | Isopropyl Alcohol | Qs |
| 6 | Purified Water | Qs |
| 7 | HPMC K l00 LV | 11.93 |
| 8 | Stearic Acid (60#) | 0.93 |
| 9 | BHA | 0.19 |
| | Functional Coating | |
| 1 | PEG 4000 | 0.78 |
| 2 | Isopropyl alcohol | Qs |
| 3 | Dichloromethane | Qs |
| 4 | Eudragit RSPO | 5.07 |
| 5 | Titanium dioxide | 0.74 |
| 6 | Ferric oxide yellow | 0.27 |

### Process for Preparation

### A) Intragranular Application of Matrixing Agent (as Per Example 1)

### B) Extragranular Application of Matrixing Agent (as Per Example 1)

### C) Coating by pH Independent Polymer

1) Dissolve PEG 4000 in approx 75% quantity of isopropyl alcohol: dichloromethane mixture with stirring.

2) Add Eudragit RSPO in step 1) with stirring.

3) Disperse the titanium dioxide and ferric oxide yellow in remaining quantity of isopropyl alcohol: dichloromethane mixture.

4) Mix the solution of step 2) and 3) for 10-20 minutes with stirring.

5) Pass the solution through 200#

6) Perform the coating with the coating solution of step 5)

### Results for Dissolution Profile Studies for Example 1, 2 and 3

| | % Dissolved | | |
|---|---|---|---|
| Time | Example | Example | Example |
| (hr) | 1 | 2 | 3 |
| 2 | 0 | 5 | 1 |
| 8 | 24 | 57 | 32 |
| 12 | 53 | 82 | 61 |
| 18 | 85 | 98 | 89 |
| 24 | 97 | 101 | 96 |

### Example 4

| Sr. No | Ingredients | % w/w |
|---|---|---|
| 1 | Paliperidone | 3.77 |
| 2 | Lactose monohydrate | 42.14 |
| 3 | HPMC K-4M | 31.45 |
| 4 | Povidone K-30 | 3.77 |
| 5 | Isopropyl alcohol | Qs |
| 6 | Purified water | Qs |
| 7 | HPMC K 100 LV | 12.08 |
| 8 | Stearic Acid (60#) | 0.94 |
| 9 | BHA | 0.19 |
| | Functional Coating | |
| 1 | Triethyl citrate | 0.28 |
| 2 | Isopropyl alcohol | Qs |
| 3 | Dichloromethane | Qs |
| 4 | Ethylcellulose 7cps | 3.96 |
| 5 | HPMC E3LV | 1.42 |

### Process for Preparation

### A) Intragranular Application of Matrixing Agent (as Per Example 1)

### B) Extragranular Application of Matrixing Agent (as Per Example 1)

### C) Coating by pH Independent Polymer

1) Dissolve HPMC E3LV in Isopropyl alcohol with stirring.

2) Dissolve ethylcellulose and triethyl citrate in dichloromethane with stirring.

3) Mix the solution of step 1) and 2) for 10-20 minutes with stirring.

4) Pass the solution through 200#

5) Perform the coating with the coating solution of step 4).

### Example 5

| Sr. No | Ingredients | % w/w |
|---|---|---|
| 1 | Paliperidone | 0.92 |
| 2 | Lactose monohydrate | 43.99 |
| 3 | HPMC K-4M | 30.67 |
| 4 | Povidone K-30 | 3.68 |
| 5 | Isopropyl Alcohol | Qs |
| 6 | Purified Water | Qs |
| 7 | HPMC K 100 LV | 11.78 |
| 8 | Stearic Acid (60#) | 0.92 |
| 9 | BHT | 0.06 |
| | Functional Coating | |
| 1 | Eudragit RSPO | 3.87 |
| 2 | PEG 4000 | 1.66 |
| 3 | Iso Propyl Alcohol | Qs |
| 4 | Dichloromethane | Qs |
| | Color Coat | |
| 1 | HPMC E5 cps | 1.58 |
| 2 | PEG 4000 | 0.20 |
| 3 | Titanium dioxide | 0.39 |
| 4 | Talc | 0.25 |
| 5 | Iron oxide Yellow | 0.01 |
| 6 | Iron oxide Red | 0.04 |
| 7 | Purified Water | Qs |

### Process for Preparation:

A) Intragranular application of matrixing agent

1) Weigh and sift paliperidone, lactose and HPMC K-4M trough 40#.

2) Mix step 1) carefully and ensures geometric mixing.

3) Dissolve Povidone K-30 in isopropyl alcohol: purified water mixer.

4) Granulate step 2) with binding solution of step 3).

5) Dry the granules and pass the dried granules through 20#.

### B) Extragranular Application of Matrixing Agent

6) Weigh and sift HPMC K 100 LV, stearic acid (60#) and BHT through 40#.

7) Lubricate step 5) with step 6). Mix well for 5 minutes.

8) Compress the resultant blend of step 7)

### C) Coating by pH Independent Polymer

1) Dissolve PEG 4000 in Isopropyl alcohol: dichloromethane solution with stirring.

2) Add Eudragit RSPO in step 1) with stirring.

3) Mix the solution for 10-20 minutes with stirring.

4) Pass the solution through 200#.

5) Perform the coating with the coating solution of step 4)

### D) Color Coating

1) Dissolve HPMC E5 and PEG 4000 in half qty of purified water with stirring

2) Disperse titanium dioxide, talc, iron oxide red and iron oxide yellow in remaining qty of purified water in homogenizer.

3) Add step 2) in step 1) with stirring.

4) Pass the solution through 200#.

5) Perform the coating with the coating solution of step 4)

### Example 6

| Sr. No | Ingredients | % w/w |
|---|---|---|
| 1 | Paliperidone | 3.68 |
| 2 | Lactose monohydrate | 41.10 |
| 3 | HPMC K-4M | 30.67 |
| 4 | Povidone K-30 | 3.68 |
| 5 | Isopropyl alcohol | Qs |
| 6 | Purified water | Qs |
| 7 | HPMC K 100 LV | 11.78 |
| 8 | Stearic acid (60#) | 0.92 |
| 9 | BHT | 0.06 |
| | Functional Coating | |
| 1 | Eudragit RSPO | 3.87 |
| 2 | PEG 4000 | 1.66 |
| 3 | Isopropyl alcohol | Qs |
| 4 | Dichloromethane | Qs |
| | Color Coat | |
| 1 | HPMC E5 cps | 1.58 |
| 2 | PEG 4000 | 0.20 |
| 3 | Titanium dioxide | 0.39 |
| 4 | Talc | 0.25 |
| 5 | Iron oxide Yellow | 0.03 |
| 6 | Iron oxide Red | 0.01 |
| 7 | Purified water | Qs |

### Process for Preparation: (As per Example 5)

Results of Dissolution Profile Studies for Examples 4, 5 and 6

| | % Dissolved | | |
|---|---|---|---|
| Time | Example | Example | Example |
| (hr) | 4 | 5 | 6 |
| 2 | 0 | 0 | 2 |
| 8 | 23 | 14 | 29 |
| 18 | 71 | 75 | 81 |
| 24 | 84 | 92 | 92 |

We claim:

1) An extended release composition of paliperidone for oral administration comprising an active ingredient and at least one matrixing agent in a ratio of 1:1 to 1:20 respectively.

2) The composition as claimed in claim 1, wherein the in-vitro release of the active ingredient is at least 50% within 12 hours.

3) The composition as claimed in claim 1, further comprises of at least one of the pharmaceutically acceptable ingredients selected from diluent 5-95%, binder 1-25%, antioxidant 0.05-2% or lubricant 0.5-10%.

4) The composition as claimed in claim 1, wherein the said matrixing agent comprises of natural or synthetic agents selected from the group comprising polysaccharides such as xanthan, pullulan, chitosan and the like; gums like guar gum, gum arabic, gum karaya, and the like; cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylethylcellulose (HPEC), hydroxypropylmethylcellulose (HPMC) and sodium carboxymethylcellulose (Na CMC); polymeric methacrylates; carbomers as well as copolymers and/or mixtures thereof of the above agents.

5) The composition as claimed in claim 1, wherein at least one matrixing agent is intra-granular and/or extra-granular; wherein the intra-granular or extra-granular agents are same or different.

6) The composition as claimed in claim 1, additionally comprises of a pH independent polymer coating.

7) The polymer coating as claimed in claim 6, wherein the said pH independent polymer coating composition comprises of low water permeable polymer, plasticizer, opacifiers and colorants.

8) A process for the preparation of an extended release composition of paliperidone for oral administration which comprises of an active ingredient and at least one matrixing agent; wherein the process comprises of the following steps:
a) Blending paliperidone and at least one intra-granular matrixing agent to form a first blend,
b) Granulating the first blend using a binding solution to form granulates,
c) Blending the granulates obtained in step b) with at least one extra-granular matrixing agent, and
d) Compressing the second blend obtained in step c) into tablet.

9) The process as claimed in claim 8, additionally comprising a pH independent polymer coating.

10) The process as claimed in claim 8, wherein at least one intra-granular matrixing agent and atleast one extra-granular matrixing agent are the same or different.
